(19) United States
(12) Patent Application Publication
(10) Pub. No.: US 2021/0268254 A1
(43) Pub. Date: Sep. 2, 2021

(54) INFUSION CONNECTION LINE

(71) Applicant: CareFusion 303, Inc., San Diego, CA (US)

(72) Inventor: Steven ZHU, Suzhou (CN)

(21) Appl. No.: 16/806,757

(22) Filed: Mar. 2, 2020

(51) Int. Cl. A61M 39/10; A61M 5/142; A61M 39/20; A61M 39/22

(52) U.S. Cl. CPC A61M 39/10; A61M 5/142; A61M 39/20; A61M 39/22; A61M 39/24; A61M 2039/1088; A61M 2039/229; A61M 2205/583; A61M 2205/0216; A61M 2039/1083

(57) ABSTRACT

Infusion connection lines for use in infusion sets are provided. The infusion connection line includes a pump infusion tube having connectors at both ends and an indicator element for showing the installation direction within an infusion pump is disposed between the connectors. The infusion connection line is configured to connect a gravity IV set to any type of infusion pump while improving the pump infusion accuracy and minimizing tube leakage or breakage over directly connecting the gravity IV set to the infusion pump. The infusion connection line may be disconnected and disposed of after use without requiring disposal of the rest of the pump IV infusion set and/or components.

### CROSS-REFERENCES TO RELATED APPLICATIONS

N/A

### BACKGROUND

Intravenous (IV) infusion sets are used in the medical field for inserting liquid substances into a patient. IV infusion sets are typically constructed by joining multiple translucent polymeric tubing segments to multiple polymeric components. IV infusion sets for use with infusion pumps are typically dedicated to infusion pump use as a complete infusion set. Further, a dedicated infusion set is required for each brand and type of infusion pump, where a particular dedicated infusion set is not compatible with any other brand or type of infusion pump. The need to buy and stock multiple different dedicated IV infusion sets results in high costs to the health care industry.

In view of the high costs of dedicated pump infusion sets, a typical work around in the health care industry is to substitute a gravity IV set for use with an infusion pump. However, gravity IV sets are not rated or qualified for use with infusion pumps, resulting in inaccuracies during pump infusion. Gravity IV sets also pose a much higher risk for broken tubing during pump infusion, which may result in patient injury. It is desirable to provide IV infusion sets for infusion pumps with reduced costs and risks.

### SUMMARY

The present disclosure provides IV infusion sets having an infusion connection line to for insertion into infusion pumps.

In one or more embodiments, an infusion connection line is provided. The infusion connection line includes a pump infusion tube, a first connector coupled to a first end of the pump infusion tube and a second connector coupled to a second end of the pump infusion tube. The infusion connection line also includes an indicator element disposed between the first and second connectors. A portion of the pump infusion tube is configured to be disposed within an infusion pump, the infusion pump being any of a plurality of types of infusion pumps used in the health care industry. The infusion connection line is configured to couple a gravity IV infusion set to the infusion pump.

In one or more aspects, the first connector is a female luer connector. In one or more aspects, the first connector is coupled to a first connector cap. In one or more aspects, the first connector is coupled to a female stop cock assembly. In one or more aspects, the first connector is coupled to a male stop cock assembly. In one or more aspects, the first connector is coupled to a needle free connector. In one or more aspects, the first connector is coupled to the gravity IV infusion set. In one or more aspects, the second connector is a male luer connector. In one or more aspects, the second connector is coupled to a second connector cap. In one or more aspects, the second connector is coupled to an extension infusion line, the extension infusion line configured to deliver a fluid from the infusion pump to a receiving entity. In one or more aspects, the pump infusion tube has a wall thickness of 0.45 mm to 0.65 mm. In one or more aspects, the pump infusion tube has a length of 300 mm to 400 mm. In one or more aspects, the pump infusion tube is formed of a high elasticity material comprising one of silicone and thermoplastic polyurethane.

In one or more embodiments, an infusion set is provided. The infusion set includes an infusion connection line. The infusion connection line includes a pump infusion tube, a female luer connector disposed on a first end of the pump infusion tube, a male connector disposed on a second end of the pump infusion tube and an indicator element disposed on the pump infusion tube between the female and male connectors. The infusion set also includes a gravity IV set coupled to the pump infusion tube. A portion of the infusion connection line is configured to be disposed within an infusion pump, the infusion pump being any of a plurality of types of infusion pumps used in the health care industry.

In one or more aspects, the infusion set includes a female stop cock assembly coupled to the female luer connector of the pump infusion tube, wherein the gravity IV set is coupled to the female stop cock assembly. In one or more aspects, the infusion set includes a male stop cock assembly coupled to the female luer connector of the pump infusion tube, wherein the gravity IV set is coupled to the male stop cock assembly. In one or more aspects, the infusion set includes a needleless connector coupled to the female luer connector of the pump infusion tube, wherein the gravity IV set is coupled to the needleless connector. In one or more aspects, the infusion set includes an extension infusion line coupled to the male connector of the pump infusion tube, the extension infusion line configured to deliver a fluid from the infusion pump to a receiving entity. In one or more aspects, the pump infusion tube has a wall thickness of 0.45 mm to 0.65 mm, and wherein the pump infusion tube has a length of 300 mm to 400 mm. In one or more aspects, the pump infusion tube is formed of a high elasticity material comprising one of silicone and thermoplastic polyurethane.

Additional features and advantages of the disclosure will be set forth in the description below and, in part, will be apparent from the description or may be learned by practice of the disclosure. The objectives and other advantages of the disclosure will be realized and attained by the structure particularly pointed out in the written description and claims hereof as well as the appended drawings.

It is to be understood that both the foregoing general description and the following detailed description are exemplary and explanatory and are intended to provide further explanation of the disclosure as claimed.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1 depicts a schematic view of an example gravity IV set.

FIG. 2 depicts an example infusion pump.

FIG. 3 depicts an infusion connection line, according to some aspects of the disclosure.

FIG. 4 depicts an infusion connection line, according to some aspects of the disclosure.

FIG. 5 depicts an infusion connection line, according to some aspects of the disclosure.

FIG. 6 depicts an infusion connection line, according to some aspects of the disclosure.

FIG. 7 depicts an infusion connection line, according to some aspects of the disclosure.

FIG. 8 depicts an infusion connection line, according to some aspects of the disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

IV infusion sets may be formed from any combination of infusion components and tubing. Typically, the infusion components and tubing are fixedly connected together to form a disposable infusion set that is used once and then discarded. The infusion components and tubing may be formed from any suitable material (e.g., plastic, silicone, rubber).

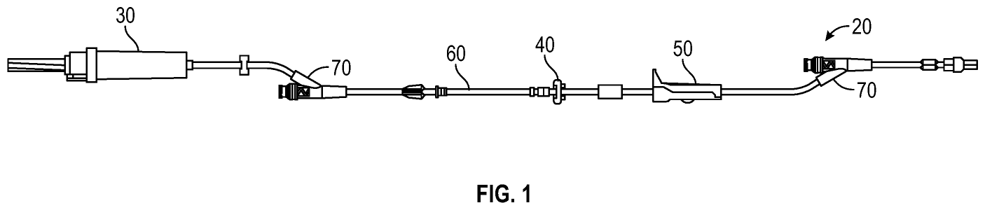

As shown in FIG. 1, an example gravity IV infusion set 20 may include a drip chamber 30, a check valve 40 and a roller clamp 50 connected together by tubing 60. Infusion set 20 may also include a Y-site 70 having a Y-shaped junction with an inlet port 72 connected to an inlet tube 62, a needleless connector 75 and an outlet port 74 connected to an outlet tube 64. The needleless connector 75 provides a port for the introduction of fluid into and/or removal of air bubbles from the infusion set 20. For example, a needleless syringe (not shown) may be connected to the needleless connector 75 in order to add medication to a saline fluid that is gravity fed from a saline bag (not shown) into the drip chamber 30. The infusion set 20 may include additional infusion components and may be formed of any combination of components and the tubing 60.

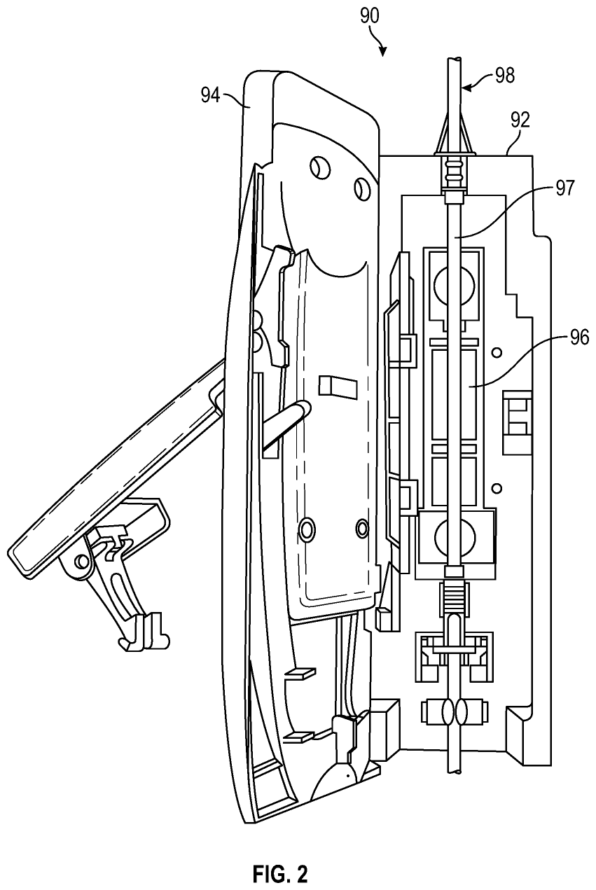

FIG. 2 shows an example infusion pump 90. The infusion pump 90 has a body 92 and a door 94. A tubing pathway 96 is disposed in the body 92. To use the infusion pump, the door 94 is opened and a tubing section 97 of an infusion set 98 is placed in the tubing pathway 96 and the door 94 is closed. The infusion pump 90 then engages and manipulates the tubing section 97 to cause fluid to be pumped through the infusion set 98.

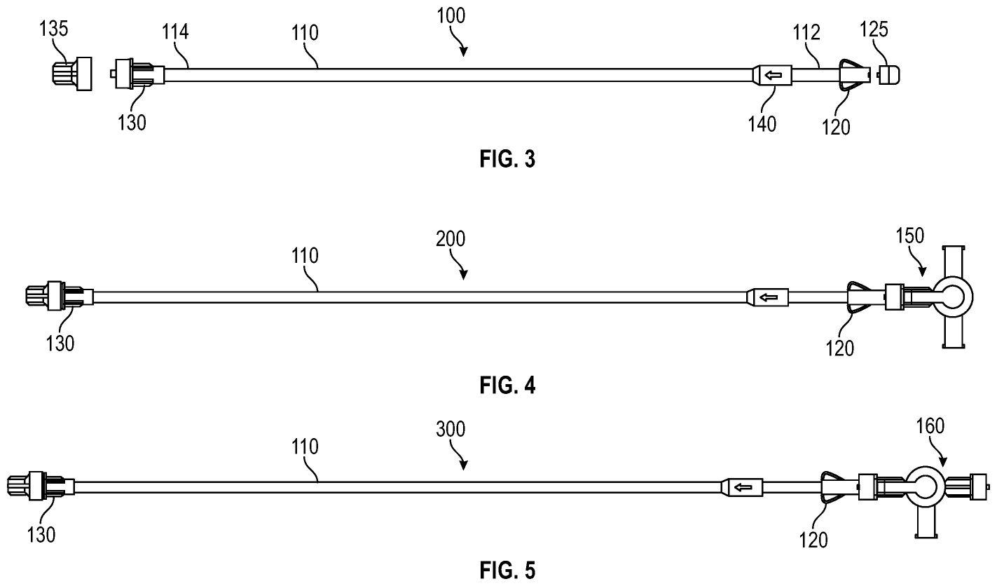

As shown in FIG. 3, an infusion connection line 100 is provided according to some aspects of the disclosure. Infusion connection line 100 includes a pump infusion tube 110. A first end 112 of the pump infusion tube 110 is coupled to a first connector 120, such as a female luer connector, for example. The first connector 120 may be coupled to a first connector cap 125, such as a female luer connector cap. A second end 114 of the pump infusion tube 110 is coupled to a second connector 130, such as a male luer lock, for example. The second connector 130 may be coupled to a second connector cap 135, such as a male luer lock cap.

An indicator element 140 may be coupled to the pump infusion tube 110 to provide a visual indication for installation within an infusion pump. In some aspects, the indicator element 140 may be printed or etched onto the pump infusion tube 110, or formed/embedded within the material of the pump infusion tube 110. A portion of the pump infusion tube 110 between the second connector 130 and the indicator element 140 is configured to be inserted or disposed within an infusion pump (e.g., infusion pump 90). The indicator element 140 is configured to indicate the proper installation direction of the infusion connection line 100 within the infusion pump.

The pump infusion tube 110 may be formed of a high elasticity material, such as silicone or TPU, for example, which is configured to be manipulated by a compression mechanism of an infusion pump. The pump infusion tube 110 may have a thickness of 0.45-0.65 mm and may have a length of 300-400 mm. Thus, the pump infusion connection line 100 is configured to provide high accuracy and stable infusion delivery regardless of which brand or type of infusion pump is used.

As shown in FIG. 4, infusion connection line 200 may include additional connection assemblies according to some aspects of the disclosure. For example, infusion connection line 200 may be infusion connection line 100 without the first connector cap 125. Infusion connection line 200 also includes a female stop cock assembly 150 (e.g., 3-way female stop cock) coupled to the first connector 120. Further, infusion connection line 200 includes the same pump infusion tube 110 as infusion connection line 100. Accordingly, infusion connection lines 100 and 200 may be used in any brand or type infusion pump.

As shown in FIG. 5, infusion connection line 300 may include additional connection assemblies according to some aspects of the disclosure. For example, infusion connection line 300 may be infusion connection line 100 without the first connector cap 125. Infusion connection line 300 also includes a male stop cock assembly 160 (e.g., 3 way male stop cock) coupled to the first connector 120. Further, infusion connection line 300 includes the same pump infusion tube 110 as infusion connection lines 100, 200. Accordingly, infusion connection lines 100, 200 and 300 may be used in any brand or type infusion pump.

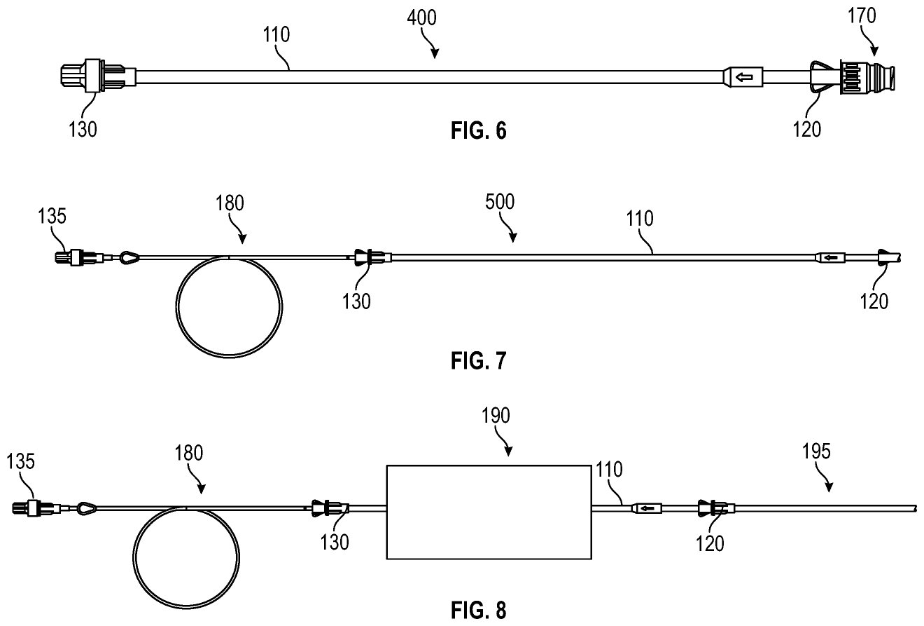

As shown in FIG. 6, infusion connection line 400 may include additional connection assemblies according to some aspects of the disclosure. For example, infusion connection line 400 may be infusion connection line 100 without the first connector cap 125. Infusion connection line 300 also includes a needle free connector 170 (e.g., needleless connector) coupled to the first connector 120. Further, infusion connection line 400 includes the same pump infusion tube 110 as infusion connection lines 100, 200, 300. Accordingly, infusion connection lines 100, 200, 300 and 400 may be used in any brand or type infusion pump.

As shown in FIG. 7, infusion connection line 500 may include additional connection assemblies according to some aspects of the disclosure. For example, infusion connection line 500 may be infusion connection line 100 without the second connector cap 135 coupled to the second connector 130. Infusion connection line 500 also includes an extension infusion line 180 coupled to the second connector 130, with the second connector cap 135 coupled to the extension infusion line 180. Further, infusion connection line 500 includes the same pump infusion tube 110 as infusion connection lines 100, 200, 300, 400. Accordingly, infusion connection lines 100, 200, 300, 400 and 500 may be used in any brand or type infusion pump.

As shown in FIG. 8, a gravity IV set 195 is coupled to infusion connection line 500 at the first connector 120. Pump infusion tube 110 is disposed within an infusion pump 190 (e.g., infusion pump 90 in FIG. 2). Thus, the gravity IV set 195 may be coupled to one or more fluid sources and a fluid flowing into the pump infusion tube 110 from the gravity IV set 195 is pumped into the extension infusion line 180 by the infusion pump 190. The extension infusion line 180 may be coupled to a receiving entity for receiving the fluid (e.g., an IV line for a patient). While infusion connection line 500 is shown to be engaged by infusion pump 190 in FIG. 8, it is understood that any of infusion connection lines 100, 200, 300, 400 may also be disposed within and/or engaged by infusion pump 190.

Because all of infusion connection lines 100, 200, 300, 400, 500 have the pump infusion tube 110 with first and second connectors 120, 130 in common, they may each be interchangeably used in any brand and type of infusion pump. Further, the configuration of the pump infusion tube 110 (e.g., material composition, thickness, size) provides for use in any brand and type of infusion pump while improving infusion accuracy and minimizing tube breakage and/or leakage in comparison to the infusion pump directly engaging a portion of a gravity IV set. In addition, the flexibility of coupling the base pump infusion tube 110 having first and second connectors 120, 130 to any of female stop cock assembly 150, male stop cock assembly 160, needle free connector 170, extension infusion line 180 and gravity IV set 195 provide for meeting a wide array of infusion needs using any infusion pump.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

1. An infusion connection line, comprising:
a pump infusion tube;
a first connector coupled to a first end of the pump infusion tube;
a second connector coupled to a second end of the pump infusion tube; and
an indicator element disposed between the first and second connectors,
wherein a portion of the pump infusion tube is configured to be disposed within an infusion pump, the infusion pump being any of a plurality of types of infusion pumps used in the health care industry, and
wherein the infusion connection line is configured to couple a gravity intravenous (IV) infusion set to the infusion pump.

2. The infusion connection line of claim 1, wherein the first connector is a female luer connector.

3. The infusion connection line of claim 2, wherein the first connector is coupled to a first connector cap.

4. The infusion connection line of claim 2, wherein the first connector is coupled to a female stop cock assembly.

5. The infusion connection line of claim 2, wherein the first connector is coupled to a male stop cock assembly.

6. The infusion connection line of claim 2, wherein the first connector is coupled to a needle free connector.

7. The infusion connection line of claim 2, wherein the first connector is coupled to the gravity IV infusion set.

8. The infusion connection line of claim 1, wherein the second connector is a male luer connector.

9. The infusion connection line of claim 8, wherein the second connector is coupled to a second connector cap.

10. The infusion connection line of claim 8, wherein the second connector is coupled to an extension infusion line, the extension infusion line configured to deliver a fluid from the infusion pump to a receiving entity.

11. The infusion connection line of claim 1, wherein the pump infusion tube has a wall thickness of 0.45 mm to 0.65 mm.

12. The infusion connection line of claim 1, wherein the pump infusion tube has a length of 300 mm to 400 mm.

13. The infusion connection line of claim 1, wherein the pump infusion tube is formed of a high elasticity material comprising one of silicone and thermoplastic polyurethane.

14. An infusion set, comprising:
an infusion connection line, comprising:
a pump infusion tube;
a female luer connector disposed on a first end of the pump infusion tube;
a male connector disposed on a second end of the pump infusion tube; and
an indicator element disposed on the pump infusion tube between the female and male connectors; and
a gravity intravenous (IV) set coupled to the pump infusion tube,
wherein a portion of the infusion connection line is configured to be disposed within an infusion pump, the infusion pump being any of a plurality of types of infusion pumps used in the health care industry.

15. The infusion set of claim 14, further comprising a female stop cock assembly coupled to the female luer connector of the pump infusion tube, wherein the gravity IV set is coupled to the female stop cock assembly.

16. The infusion set of claim 14, further comprising a male stop cock assembly coupled to the female luer connector of the pump infusion tube, wherein the gravity IV set is coupled to the male stop cock assembly.

17. The infusion set of claim 14, further comprising a needleless connector coupled to the female luer connector of the pump infusion tube, wherein the gravity IV set is coupled to the needleless connector.

18. The infusion set of claim 14, further comprising an extension infusion line coupled to the male connector of the pump infusion tube, the extension infusion line configured to deliver a fluid from the infusion pump to a receiving entity.

19. The infusion set of claim 14, wherein the pump infusion tube has a wall thickness of 0.45 mm to 0.65 mm, and wherein the pump infusion tube has a length of 300 mm to 400 mm.

20. The infusion set of claim 14, wherein the pump infusion tube is formed of a high elasticity material comprising one of silicone and thermoplastic polyurethane.
